(19) United States
(12) Patent Application Publication
(10) Pub. No.: US 2009/0203771 A1
(43) Pub. Date: Aug. 13, 2009

(54) NOVEL INTERMEDIATE FOR HALICHONDRIN B ANALOG SYNTHESIS AND NOVEL DESULFONYLATION REACTION USED FOR THE INTERMEDIATE

(75) Inventors: Kazato INANAGA, Kasumi-shi (JP); Manabu Kuboto, Kasumi-shi (JP); Akio Kayano, Tsuchiura-shi (JP); Katsuya Tagami, Tsuchiura-shi (JP)

Correspondence Address: Clark & Elbing LLP / Eisai, 101 Federal Street, Suite 1500, Boston, MA, 02110, US

(73) Assignee: Eisai R&D Management Co., Ltd., Tokyo (JP)

(21) Appl. No.: 12/271,731

(22) Filed: Nov. 14, 2008

Related U.S. Application Data
(60) Provisional application No. 60/988,496, filed on Nov. 16, 2007.

(30) Foreign Application Priority Data
Nov. 16, 2007 (JP) P2007-298074

(51) Int. Cl. A61K 31/365; C07F 15/00

(52) U.S. Cl. 514/450; 549/267; 546/2

(57) ABSTRACT

The present invention provides a novel method for producing a compound represented by formula (III) shown below, which comprises treating a compound represented by formula (I) shown below with a trivalent chromium compound and at least one kind of metal selected from the group consisting of manganese and zinc in a solvent in the presence of a ligand represented by formula (II) shown below, and the present invention further provides the novel compound represented by formula (I).

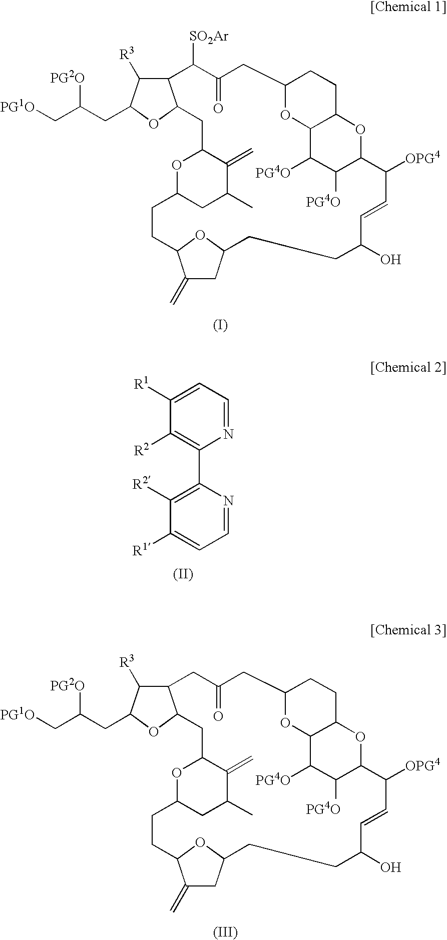

This application claims priority on Japanese Patent Application No. 2007-298074 filed on Nov. 16, 2007, in Japan and U.S. Patent Provisional Application No. 60/988,496 filed on Nov. 16, 2007, in the U.S., the disclosures of which are incorporated by reference herein in their entirety.

### TECHNICAL FIELD

The present invention relates to a novel compound represented by formula (I) shown below and a method for producing the same, and a method for producing a compound represented by formula (III) shown below from the compound (I), especially a novel desulfonylation reaction.

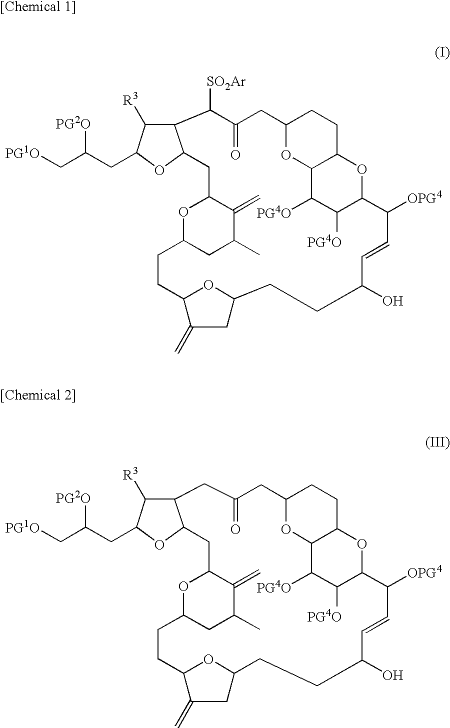

### BACKGROUND ART

Halichondrin B is a natural product having potent anti-tumor activity, which was isolated first from the marine sponge Halichondria okadai and subsequently discovered in Axinella sp., Phakellia carteri and Lissondendryx sp. The complete synthesis of Halichondrin B was made public in 1992 (Non-Patent Document 1 and Patent Document 1). Halichondrin B shows tubulin polymerization, microtubule aggregation, beta-tubulin crosslinking, binding of GTP and Vinblastine to tubulin, and tubulin-dependent GTP hydrolysis in vitro, and also shows anti-tumor activity both in vitro and in vivo.

Analogues of Halichondrin B having pharmaceutical activity such as anti-tumor activity or anti-mitosis activity (mitosis inhibitory activity) and a synthesis method thereof have also been made public (see, for example, Patent Document 2). Patent Document 2 discloses, as an analogue of Halichondrin B having pharmaceutical activity, a compound B-1939 shown below and a synthesis method thereof.

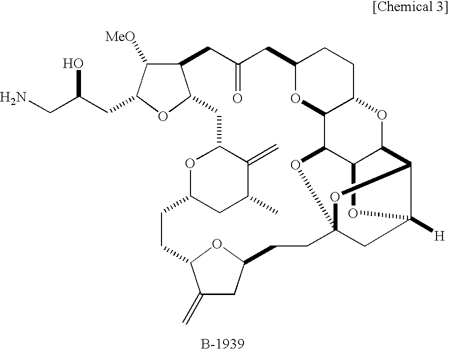

### [Patent Document 1]

Specification of U.S. Pat. No. 5,338,865

### [Patent Document 2]

Pamphlet of International Publication No. WO 2005/118565

### [Non-Patent Document 1]

Aicher, T. D. et al., J. Am. Chem. Soc., 114: 3162-3164 (1992)

### [Non-Patent Document 2]

Protecting Groups in Organic Synthesis, T. W. Greene and P. G. M. Wuts, 3^{rd} edition, John Wiley & Sons, 1999

### [Non-Patent Document 3]

P. J. Kocienski, Protecting Groups, Thieme, 1994

### [Non-Patent Document 4]

Namba, K.; Kishi, Y. J. Am. Chem. Soc. 2005, 127, 15382

### DISCLOSURE OF THE INVENTION

One of key steps in the synthesis path of B-1939 described in Patent Document 2 is the step of cyclizing an intermediate ER-118049 by intramolecular coupling to obtain ER-118047/048 (paragraph [00206] of Patent Document 2). This ER-118049 is obtained by desulfonylation of ER-804030 (paragraph [00205] of Patent Document 2). In the desulfonylation reaction described in Patent Document 2, SmI₂ is used as a reducing agent. However, SmI₂ is expensive and is not a compound which is easily available in large quantities, and also SmI₂ is not easy to handle since it is very unstable when exposed to oxygen in the air. Although desulfonylation reactions using reducing agents such as Na—Hg amalgam, Al—Hg amalgam, Mg-alcohol, Zn, and Zn—Cu are known, the desulfonylation reaction of ER-804030 using reducing agents such as Mg-alcohol, Zn, and Zn—Cu does not provide good results.

Therefore, there is a need to develop, as the reaction path for obtaining ER-118047/048 from ER-804030, a novel reaction path which can reduce a sulfonyl group under mild reaction conditions using a reducing agent, which is easily available and is also easily handled, and also can perform intramolecular coupling between a vinyl iodide group and an aldehyde group in good yields; an intermediate compound to be used for the reaction path; and a novel desulfonylation reaction to be used in the reaction path.

The present inventors have found that, using a compound represented by formula (I) shown below, which is synthesized by intramolecular coupling of a compound represented by formula (IV) shown below, as a novel intermediate, a compound represented by formula (III) shown below can be obtained in high yield by the desulfonylation reaction of the intermediate under mild reaction conditions. This reaction path can serve as a novel synthesis path which is useful to synthesize B-1939 described in the pamphlet of International Publication No. WO 2005/118565.

The present inventors have found that a compound represented by formula (III) shown below can be obtained in high yield under mild reaction conditions by desulfonylation of the compound represented by formula (I) through treatment with a trivalent chromium compound and at least one kind of metal selected from the group consisting of manganese and zinc in a solvent in the presence of a ligand of formula (II) shown below. Thus, the present invention has been completed.

Cr(III)X₃ is preferably used as the trivalent chromium compound. In the formula, X represents a halogen atom and X is preferably a chlorine (Cl) or bromine (Br) atom.

It is particularly preferred to use at least one kind selected from the group consisting of CrCl₃ anhydride, CrCl₃.6H₂O and CrCl₃.3THF as the trivalent chromium compound used in the present invention.

It is preferred that R¹ and R^{1′} as ligands of formula (II) shown below used in the present invention represent t-butyl, phenyl, or nonyl, and R² and R^{2′} represent a hydrogen atom, or R² and R^{2′} are preferably combined to form a fused ring together with a pyridine ring to which they are attached.

It is preferred to further add a metallocene compound selected from the group consisting of Ti, Zr and Hf compounds, containing a cyclopentadienyl ring for the desulfonylation reaction of the present invention. The amount of a trivalent chromium compound to be used can be decreased by using the metallocene compound.

The desulfonylation reaction of the present invention proceeds under mild conditions. The desulfonylation reaction is preferably carried out at a temperature of 20 to 30° C.

The solvent used for the desulfonylation reaction of the present invention is particularly preferably a mixture of one or more kinds selected from the group consisting of tetrahydrofuran, dimethoxyethane, methyl t-butylether, dimethylformamide, methanol, and acetonitrile.

The present invention will be described in more detail below.

A novel reaction path, which has been developed this time by the present inventors, is shown in Scheme 1.

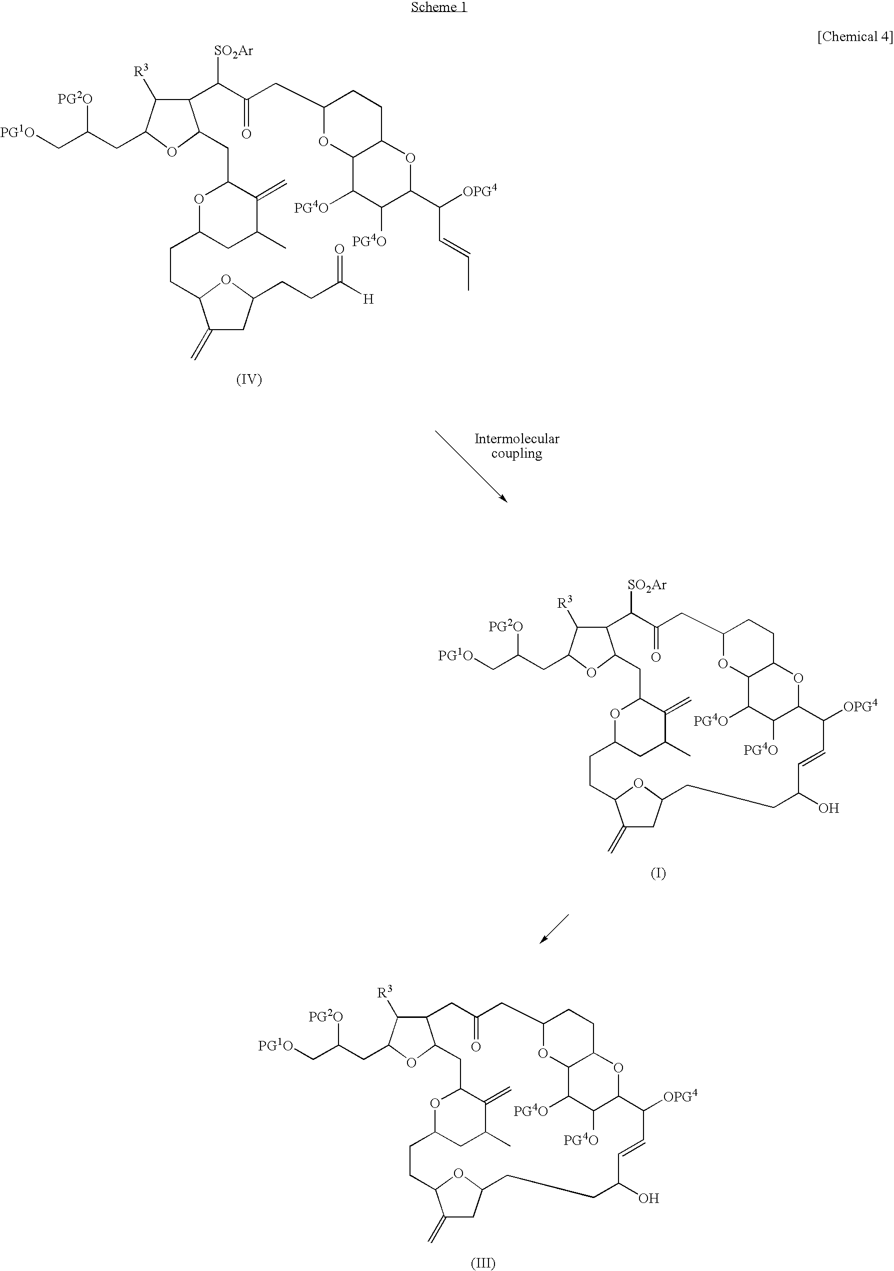

According to the present invention, as shown in Scheme 1, a compound (I) is obtained by intramolecular coupling of a compound (IV) and a compound (III) is obtained by desulfonylation of the compound (I). One example of the compound (IV) includes ER-804030 disclosed in paragraph [00203] of the pamphlet of International Publication No. WO 2005/118565. In that case, the compound (III) obtained by the reaction path of the aforementioned Scheme 1 is ER-118047/048 described in paragraph [00205] of the pamphlet of International Publication No. WO 2005/118565.

An intermediate in the aforementioned Scheme 1 is a compound represented by formula (I) shown below.

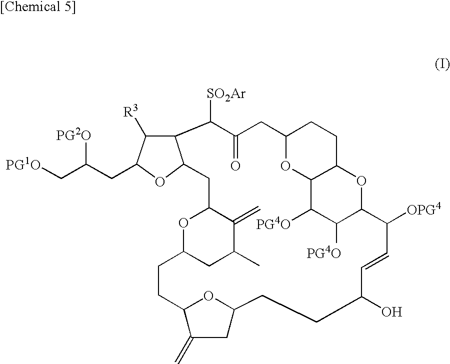

Meanings of symbols R³, Ar, PG¹, PG² and PG⁴ in formula (I) will be explained below, and symbols R³, Ar, PG¹, PG² and PG⁴ in formulas (IV) and (III) have the same meanings.

In formula (I), R³ represents R or OR, R represents a hydrogen atom, a halogen atom, a C₁₋₄ halogenated aliphatic group, benzyl, or a C₁₋₄ aliphatic group. Examples of the halogen atom include fluorine, chlorine, bromine and iodine atoms and, among these atoms, fluorine and chlorine atoms are preferred. Examples of the C₁₋₄ halogenated aliphatic group include, but are not limited to, fluoromethyl, trifluoromethyl, and chloromethyl. Examples of the C₁₋₄ alkyl group include methyl, ethyl, n-propyl, isopropyl, n-butyl, sec-butyl, and t-butyl. A methoxy (OMe) group is particularly preferred as R³.

In formula (I), Ar represents a substituted or unsubstituted aryl group, or a substituted or unsubstituted heteroaryl group.

The aryl group represented by Ar is preferably an aromatic hydrocarbon group having 6 to 10 carbon atoms, and examples thereof include a phenyl group and a naphthyl group. The aryl group may or may not further have one or more substituent groups, and examples of the substituent groups include, but are not limited to, a substituted or unsubstituted alkyl group, a substituted or unsubstituted aryl group, a halogen atom such as a fluorine or chlorine atom, and C₁₋₆ alkoxy. Specific examples of Ar include a phenyl group, a 2-methylphenyl group, a 4-methylphenyl group, and a naphthyl group. Ar is particularly preferably a phenyl group.

Ar may be a substituted or unsubstituted heteroaryl group. In this case, the substituent group includes the same substituent groups as those of the aryl group. Examples of the heteroaryl group include a quinolinyl group.

PG¹, PG² and PG⁴ in formula (I) each independently represents a protective group of a hydroxyl group. A suitable protective group of the hydroxyl group is known in this field and includes protective groups described in “Protecting Groups in Organic Synthesis, T. W. Greene and P. G. M. Wuts, 3^{rd} edition, John Wiley & Sons, 1999”. In specific embodiments, PG¹, PG² and PG⁴ are independently selected, as a group containing the oxygen atom to which they are attached, from esters, ethers, silylethers, alkylethers, aralkylethers, and alkoxyalkylethers. Examples of the esters include formates, acetates, carbonates, and sulfonates. Specific examples thereof include formate, benzoylformate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, (trimethylacetyl)pivaloate, crotonate, 4-methoxy-crotonate, benzoate, p-phenylbenzoate, 2,4,6-trimethylbenzoate, or carbonates (for example, methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonates). Examples of the silylethers include trimethylsilyl, triethylsilyl, t-butyldimethylsilyl, t-butyldiphenylsilyl, triisopropylsilyl, and other trialkylsilyl ethers. Examples of the alkylethers include methyl, benzyl, p-methoxybenzyl, 3,4-dimethoxybenzyl, trityl, t-butyl, allyl, and allyloxycarbonyl ethers or a derivative group thereof. Examples of the alkoxyalkylethers include ethers such as methoxymethyl, methylthiomethyl, (2-methoxyethoxy)methyl, benzyloxymethyl, β-(trimethylsilyl)ethoxymethyl, and tetrahydropyranyl ethers. Examples of the arylalkylethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers. In a specific aspect, one or more of PG¹, PG² and PG⁴ are silylethers or aryl alkyl ethers. In another aspect, at least one of PG¹, PG² and PG⁴ is t-butyldimethylsilyl or benzoyl. In a particularly preferred aspect, PG¹, PG² and PG⁴ represent t-butyldimethylsilyl.

According to another aspect, PG¹ and PG², and two PG⁴ may form a diol protective group such as acetal or ketal together with the oxygen atom to which they are attached. Examples of the diol protective group include methylene, ethylidene, benzylindene, isopropylidene, cyclohexylidene, cyclopentylindene, a silylene derivative group such as di-t-butylsilylene or 1,1,3,3-tetraisopropylsiloxanylidene, cyclic carbonate, and cyclic boronate. Regarding a method for addition or removal of a protective group of a hydroxyl group, and additional protective groups, please refer to the aforementioned “Protecting Groups in Organic Synthesis”, T. W. Greene et al.; and “Protecting Groups, Thieme, 1994”, P. J. Kocienski.

<Intramolecular Coupling Reaction: Synthesis of Compound of Formula (I) from Compound of Formula (IV)>

As shown in Scheme 1, a compound of formula (I) (hereinafter referred to as “compound I”) can be synthesized by intramolecular coupling of a compound of formula (IV) (hereinafter referred to as “compound IV”).

The compound IV is available based on the synthesis method described in detail in WO2005/118565. A compound IV having various protective groups of a hydroxyl group can be synthesized by substituting the protective group of the hydroxyl group with a desired protective group in the synthesis method.

A compound I is obtained by intramolecular coupling of an aldehyde group and a vinyl iodide group in the compound IV. This coupling reaction can be carried out using Ni(II)—Cr(II) as described in the aforementioned Patent Document 1 and paragraph [00206] of WO2005/118565.

<Desulfonylation Reaction: Synthesis of Compound of Formula (III) from Compound I>

As shown in Scheme 1, a compound of formula (III) (hereinafter referred to as “compound III”) can be synthesized by desulfonylation of a compound I. The present inventors have found that desulfonylation proceeds under mild conditions to obtain a compound III in a high yield by treating a compound I with a trivalent chromium compound and at least one kind of metal selected from the group consisting of manganese and zinc in the presence of a specific ligand.

That is, desulfonylation of a compound I can be carried out by treating the compound I with a trivalent chromium compound and at least one kind of metal selected from the group consisting of manganese and zinc in a solvent in the presence of a ligand represented by formula (IT) shown below:

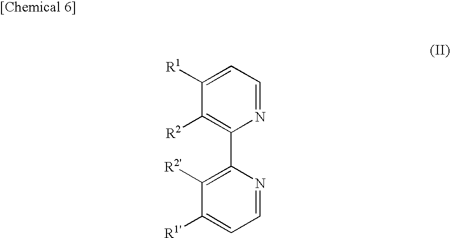

Specifically, this treatment can be carried out by mixing an organosulfone compound, a trivalent chromium compound, manganese metal and/or zinc metal as raw materials in a solvent in the presence of a ligand of formula (II).

In formula (II) shown above, R¹ and R^{1′} each independently represents a C₃₋₁₂ alkyl group, or an unsubstituted or substituted phenyl group. The C₃₋₁₂ alkyl group includes a straight-chain, branched or cyclic alkyl group and examples thereof include propyl, butyl, pentyl, hexyl, heptyl, octyl, nonyl, decyl and dodecyl groups, and isomers thereof. Among these groups, t-butyl and nonyl groups are particularly preferred. Examples of the substituent group in a phenyl group include, but are not limited to, halogen atoms (for example, fluorine and chlorine atoms), C₁₋₁₂ alkyl groups (for example, straight-chain, branched and cyclic alkyl groups), and C₁₋₆ alkoxy groups (for example, methoxy, ethoxy, propoxy and butoxy groups). A particularly preferred unsubstituted or substituted phenyl group is an unsubstituted phenyl group.

R² and R^{2′} each independently represents a hydrogen atom or a C₁₋₆ alkyl group. The C₁₋₆ alkyl group includes a straight-chain, branched or cyclic alkyl group, and examples thereof include methyl, ethyl, propyl, butyl, pentyl and hexyl groups, and isomers thereof.

R² and R^{2′} may be combined to form a fused ring together with two pyridine rings to which they are attached. Examples of the fused ring include 1,10-phenanthroline, 5,6-dimethyl-1,10-phenanthroline, 5,6-dihydro-1,10-phenanthroline, and 4,7-diphenyl-1,10-phenanthroline.

Among the compounds represented by formula (II) (hereinafter referred to as “ligand II”), 4,4′-di-t-butyl-2,2′-bipyridyl, 4,7-diphenyl-1,10-phenanthroline, 4,4′-diphenyl-2,2′-bipyridyl and 4,4′-dinonyl-2,2′-bipyridyl are particularly preferred.

The solvent used for the desulfonylation reaction may be any solvent as long as it does not inhibit the desulfonylation reaction. These solvents can be used alone, or two or more kinds of them can be used in combination. Examples of preferred solvents include tetrahydrofuran (THF), dimethoxyethane (DME), methyl t-butylether (MTBE), dimethylformamide (DMF), methanol, and acetonitrile, and it is preferred to use one kind of solvent selected from these solvents, or a mixture of two or more kinds selected from them.

A known trivalent chromium compound can be used for the desulfonylation reaction of the present invention. As the trivalent chromium compound, a known organic chromium compound and a known inorganic chromium compound can be used, and an inorganic chromium compound is preferred. A particularly preferred trivalent chromium compound is a chromium(III) halide represented by Cr(II)X₃ (wherein X represents a halogen atom). X is preferably Cl (chlorine) or Br (bromine). Particularly preferred trivalent chromium compounds are CrCl₃ anhydride and CrCl₃.6H₂O. CrCl₃.3THF is also preferred.

In the desulfonylation reaction of the present invention, one or more kinds of metals selected from manganese and zinc are used together with the trivalent chromium compound. Since the reaction rate can be enhanced, powdered manganese and powdered zinc are preferably used.

In order to obtain a desulfonylated product in a high yield, the trivalent chromium compound may be used in the amount of 1 molar equivalent or more, particularly 1 to 10 molar equivalents, and preferably 2 to 5 molar equivalents, based on the organosulfone compound as a starting material. However, the amount of the trivalent chromium compound is not limited to the above range. As explained hereinafter, the amount of the trivalent chromium compound can be remarkably decreased by adding a small amount of a metallocene compound selected from zirconocene dichloride.

The manganese metal and/or zinc metal to be used together with the trivalent chromium compound may be used in the amount of 1 molar equivalent or more, particularly 1 to 100 molar equivalents, preferably from 3 to 30 molar equivalents, and more preferably 5 to 20 molar equivalents, based on the organosulfone compound as a starting material. Usually, it is preferred to use manganese metal and/or zinc metal which have larger molar equivalents than those of the trivalent chromium compound to be used.

The desulfonylation reaction of the present invention can be carried out at a temperature of 5 to 50° C., and particularly preferably 20 to 30° C., but the reaction temperature is not specifically limited. A significant feature of the desulfonylation reaction of the present invention is that it can be carried out at room temperature. However, the desulfonylation reaction can also be carried out at a temperature which is higher or lower than room temperature (20 to 30° C.). The objective desulfonylated product is obtained by mixing a reaction mixture with stirring at a desired reaction temperature.

The desulfonylation reaction is preferably carried out under the atmosphere of an inert gas, for example, nitrogen or argon.

Furthermore, the present inventors have found that, by using a metallocene compound together with a trivalent chromium compound in the desulfonylation reaction of the present invention, a desulfonylation reaction product is obtained in a high yield even when the amount of the trivalent chromium compound to be used is less than 1 molar equivalent based on the organosulfone compound. For example, by using zirconocene dichloride (Cp₂ZrCl₂) in the amount of 1 molar equivalent based on the organosulfone compound, a desulfonylated product is obtained in a high yield even when the trivalent chromium compound is used in the amount of less than 1 molar equivalent, for example, 0.2 molar equivalents, based on the organosulfone compound. Therefore, the amount of the trivalent chromium compound can be remarkably decreased by adding the metallocene compound. Each amount of the metallocene compound and the trivalent chromium compound to be used for the desulfonylation reaction can be adjusted to a suitable amount so as to obtain a desired desulfonylated product in a desired yield.

Examples of the metallocene compound include compounds having a cyclopentadienyl ring of a transition metal selected from the group consisting of Group 4 transition metals (Ti, Zr, and Hf) of the Periodic Table. These compounds are known and include, for example, various metallocene compounds described in Japanese Unexamined Patent Application, First Publication No. 2006-63158 (paragraphs [0024] to [0031]). Examples of the metallocene compound include bis(cyclopentadienyl)zirconium dichloride; a bis(mono- or polyalkyl substituted cyclopentadienyl)zirconium dichloride such as bis(methylcyclopentadienyl)zirconium chloride or bis(pentamethylcyclopentadienyl)zirconium chloride; bis(indenyl)zirconium dichloride; a zirconium compound such as a bis(mono- or polyalkyl substituted indenyl)zirconium dichloride; and titanium and hafnium compounds, each having a chemical structure in which a zirconium atom of these compounds is replaced by a titanium or hafnium atom. As the metallocene compound used for the desulfonylation reaction of the present invention, a Zr compound is preferred and bis(cyclopentadienyl)zirconium dichloride is particularly preferred.

According to the desulfonylation reaction of the present invention, since a desulfonylated product can be obtained in a high yield under conditions at room temperature, desirable results can be obtained even when an unstable compound is used as a starting material. Since this reaction can be carried out only by stirring all raw materials in a solvent at room temperature, it is easy to control the reaction conditions.

### BEST MODE FOR CARRYING OUT THE INVENTION

The present invention will be described in detail with reference to Examples. The present invention is not limited to the following Examples and modifications can be made without departing from the spirit or scope of the present invention.

ER-804030 used in the following Examples was synthesized in accordance with the method described in the Examples of the pamphlet of International Publication No. WO 2005/118565. Commercially available products were used as a ligand II, a trivalent chromium compound, manganese metal, zirconocene dichloride and a solvent in the reaction. In the Examples, THF denotes tetrahydrofuran, DME denotes dimethoxyethane, ACN denotes acetonitrile, HPLC denotes high-performance liquid chromatography, TLC denotes thin-layer chromatography, TBS denotes t-butyldimethylsilyl, and Cp denotes a cyclopentadienyl group, respectively.

A CrCl₃/4,4′-di-t-butyl-bipyridyl catalyst and a NiCl₂/2,9-dimethyl-1,10-phenanthroline catalyst used in the following Examples were prepared in accordance with the method described in Namba, K.; Kishi, Y. J. Am. Chem. Soc. 2005, 127, 15382.

The NiCl₂/2,9-dimethyl-1,10-phenanthroline catalyst was prepared in the following manner.

In a reaction vessel, a NiCl₂-DME complex (660 mg, 3.0 mmol, 1.0 molar equivalent), 2,9-dimethyl-1,10-phenanthroline (Neocuproine; 659 mg, 3.0 mmol, 1.0 molar equivalent) were charged after weighing and, after the reaction vessel was depressurized, the atmosphere in the reaction vessel was replaced by nitrogen. Then, anhydrous acetonitrile (40 ml) was added and the contents were well mixed. Ultrasonic waves were applied to the resultant reaction solution for one minute, followed by standing for 20 minutes. The supernatant was removed and a yellow precipitate was dried under reduced pressure to obtain 668 mg of a yellow powder (yield: 65.9%).

### Example 1

### Production Example 1 of ER-413207

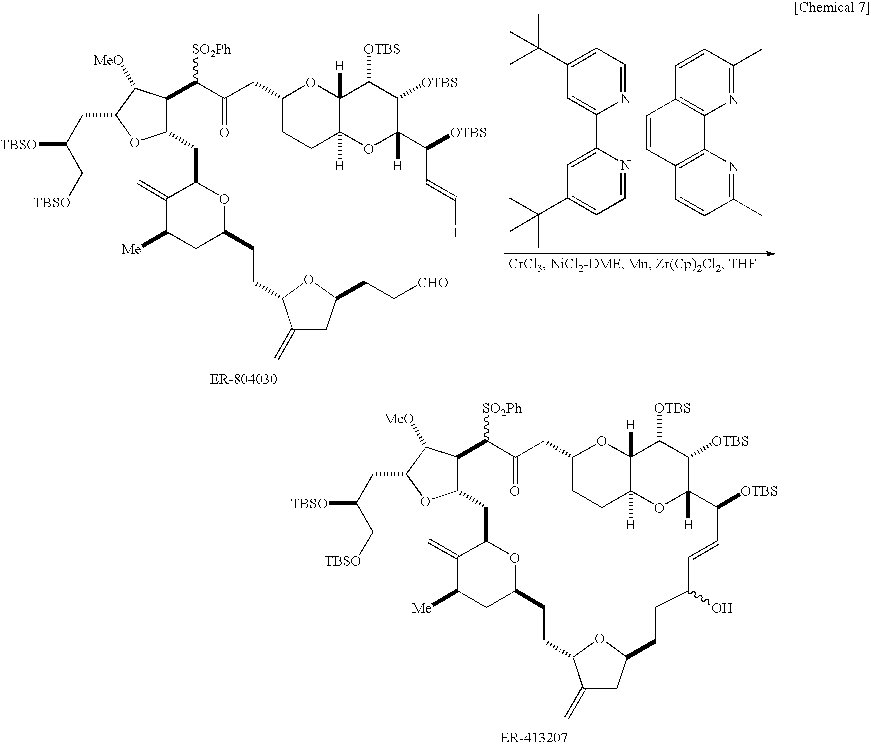

4,4′-di-t-butyl-bipyridyl (3.4 mg, 0.0126 mmol, 0.10 molar equivalents), CrCl₃ (2.0 mg, 0.0126 mmol, 0.10 molar equivalents), a manganese powder (27.7 mg, 0.504 mmol, 4.0 molar equivalents) and bis(cyclopentadienyl)zirconium dichloride (55.2 mg, 0.189 mmol, 1.5 molar equivalents) were weighed and placed in a reaction vessel, and then the atmosphere in the reaction vessel was replaced by a nitrogen gas. In the reaction vessel, THF (2.0 ml, anhydrous, free from stabilizer) was added, followed by stirring at room temperature for 90 minutes. Under a nitrogen atmosphere, 2,9-dimethyl-1,10-phenanthroline (2.6 mg, 0.0126 mmol, 0.10 molar equivalents) and NiCl₂-DME complex (2.8 mg, 0.0126 mmol, 0.10 molar equivalents) were added, followed by stirring at room temperature for 30 minutes. To the resultant reaction solution, a THF solution (10 ml) of ER-804030 (200 mg) was added, followed by stirring at room temperature for 2 hours. After confirming the completion of the reaction by HPLC, hexane (6.0 ml) was added to the reaction solution and the supernatant was transferred to a separating funnel. The organic layer was washed with an aqueous 10% citric acid solution (6.0 ml) to isolate the organic layer. The aqueous layer was reextracted with hexane (3.0 ml) and the hexane layer was mixed with the organic layer. Hexane (2.0 ml) was added to the organic layer and, after washing with 10% saline (4.0 ml), the organic layer was concentrated to obtain 213 mg of an ER-413207 crude product. The crude product was purified by column chromatography using silica gel (17 g) (eluate: heptane/ethyl acetate) to obtain 152.5 mg (yield: 82.8%) of a purified product as a white solid.

TLC (Hexane/EtOAc=4/1), Rf=0.2, 0.4, color coupler: anisic aldehyde

¹H NMR (400 MHz, CDCl₃) 7.96 (dd, 1H, J=8.8, 1.6 Hz), 7.82 (d, 1H, J=7.2 Hz), 7.68 (t, 1H, J=7.2 Hz), 7.59 (d, 1H, J=8.4), 7.55 (d, 1H, J=7.6 Hz), 6.10-5.95 (m, 1H), 5.80-5.65 (m, 1H), 5.05-4.90 (m, 2H), 4.85-4.70 (m, 4H), 4.55-4.40 (m, 2H), 4.35-4.25 (m, 1H), 4.25-4.12 (m, 3H), 4.12-3.95 (m, 2H), 3.95-3.75 (m, 5H), 3.75-3.35 (m, 9H), 3.21 (s, 3H), 3.30-2.45 (m, 6H), 2.25-2.00 (m, 5H), 2.00-1.20 (m, 9H), 1.10-1.00 (m, 3H), 1.00-0.80 (m, 45H), 0.20-0.00 (m, 30H) MS m/z 1484 (M+Na)+ (ESI Positive)

### Example 2

### Production Example 2 of ER-413207

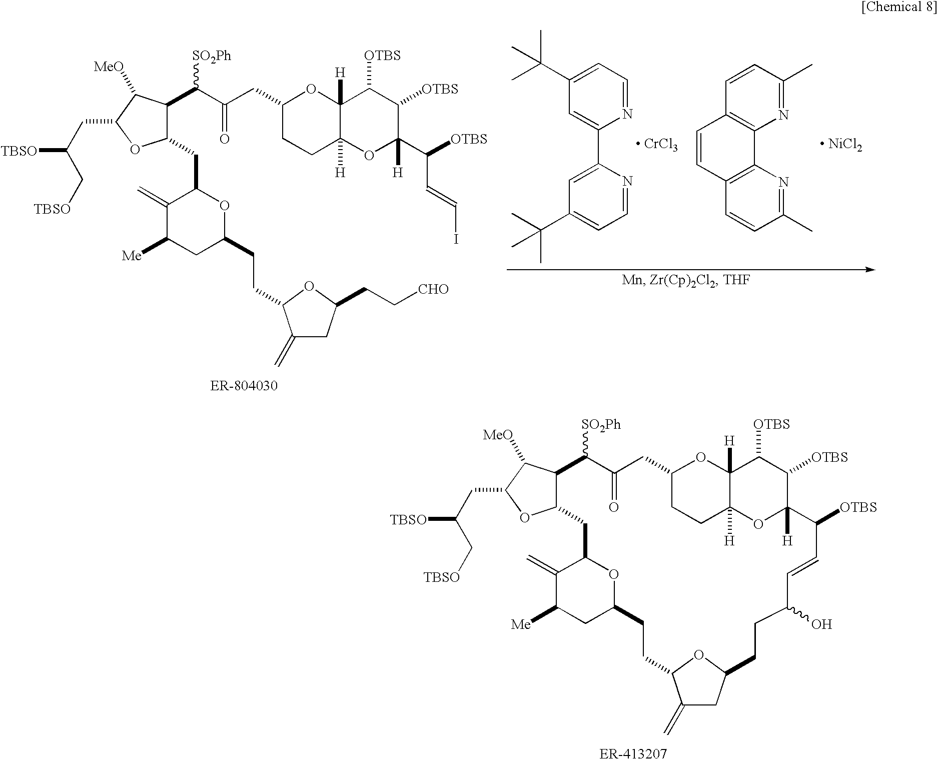

Under a nitrogen atmosphere, a CrCl₃/4,4′-di-t-butyl-bipyridyl catalyst (5.4 mg, 0.0126 mmol, 0.10 molar equivalents), a NiCl₂/2,9-dimethyl-1,10-phenanthroline catalyst (4.3 mg, 0.0126 mmol, 0.10 molar equivalents), a manganese powder (27.7 mg, 0.504 mmol, 4.0 molar equivalents) and bis(cyclopentadienyl)zirconium dichloride (55.2 mg, 0.189 mmol, 1.5 molar equivalents) were weighed and placed in a 50 ml recovery flask and anhydrous THF (8.0 ml, 40 μl/mg, free from stabilizer, dried over molecular sieves 4A) was added, and then the resultant reaction solution was stirred for 30 minutes. In the reaction solution, an anhydrous THF solution (4.0 ml) of ER-804030 (200 mg, 0.126 mmol) was added and the resultant mixture was stirred under a nitrogen atmosphere at room temperature (25° C.) for 6 hours. After confirming the completion of the reaction by HPLC, the reaction solution was diluted with ethyl acetate (100 ml) under air. The resultant solution was filtered through silica gel (16 g) and the silica gel was rinsed in turn with ethyl acetate (40 ml) and heptane (40 ml). The filtrate and the wash were combined and concentrated to obtain an ER-413207 crude product in a yield of 91.2% (HPLC quantitative value). The crude product was purified by column chromatography using silica gel (11 g) (eluate: heptane/ethyl acetate) to obtain 159.6 mg (yield: 86.7%) of ER-413207 as a white solid.

### Example 3

### Production Example 3 of ER-413207

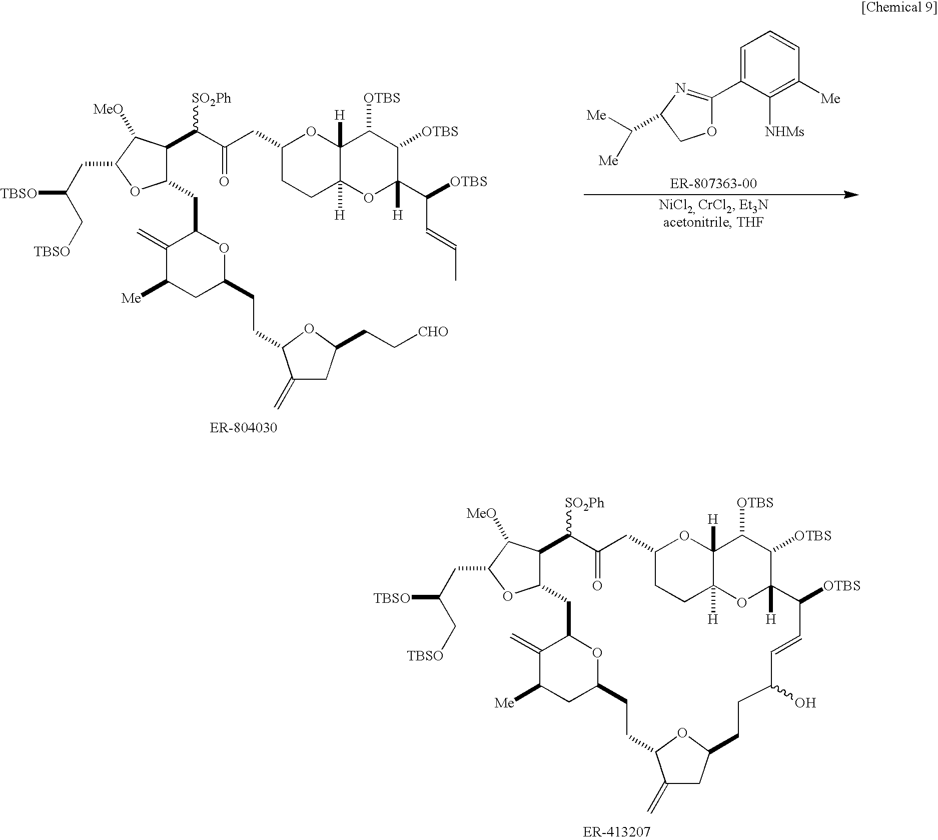

This Example was carried out with reference to an example (paragraph [00206]) described in the pamphlet of International Publication No. WO 2005/118565.

ER-807063 (1.9 g, 6.40 mmol) was weighed and placed in a reaction vessel, acetonitrile (27 ml) was added and dissolved. In the resultant reaction solution, CrCl₂ (800 mg, 6.51 mmol) and triethylamine (0.8 ml, 6.00 mmol) were added, followed by stirring at about 30° C. for 3 hours. The reaction vessel was cooled to 15° C. and NiCl₂ (100 mg, 0.771 mmol) was introduced, and then a preliminarily prepared THF-ACN mixed solution (THF/ACN=84/16, 31 mL) of ER-804030 was added dropwise to the reaction solution over 30 minutes. After the completion of the addition of the ER-804030 solution, the reaction mixture was stirred at a temperature within a range from 15 to 21° C. for 3 hours while gradually heating and heptane (25 ml) was introduced into the reaction mixture. The reaction mixture was filtered on a celite pad and then the celite pad was rinsed with heptane (10 ml) and acetonitrile (10 ml). The upper layer (heptane layer) of the resultant solution was isolated and the lower layer (acetonitrile layer) was extracted with heptane (30 ml). The combined heptane layer was washed twice with acetonitrile (10 ml) and then concentrated to obtain 766 mg of an ER-413207 crude product. This crude product was purified by silica gel column chromatography (eluate:heptane/ethyl acetate) to obtain 673.3 mg (76.7%, 0.460 mmol) of ER-413207 as a colorless solid.

### Example 4

### Production Example 4 of ER-413207

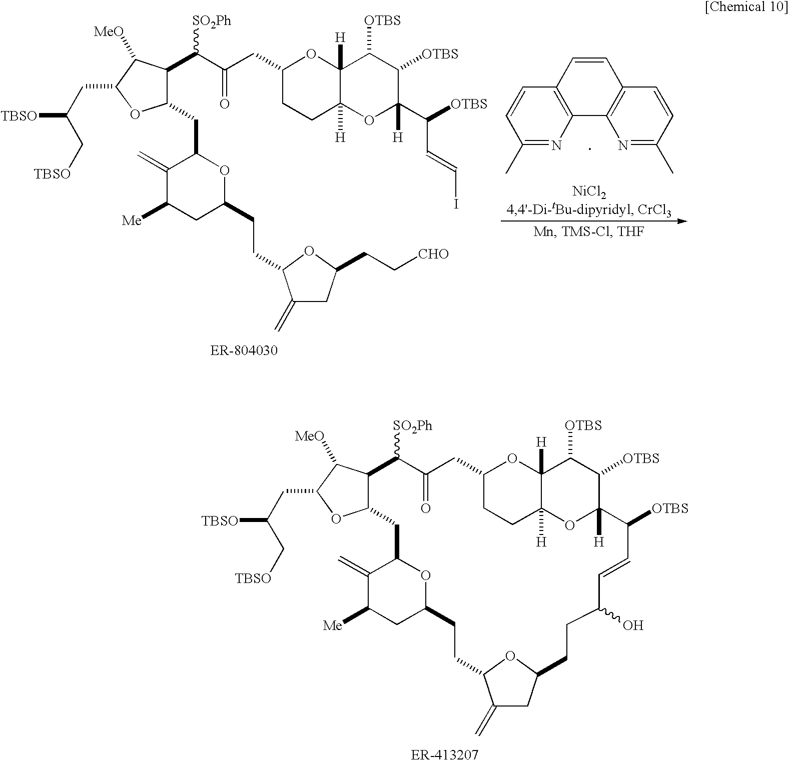

4,4′-di-t-butyl-bipyridyl (3.4 mg, 0.0126 mmol, 0.10 molar equivalents), CrCl₃ (2.0 mg, 0.0126 mmol, 0.10 molar equivalents) and a manganese powder (27.7 mg, 0.504 mmol, 4.0 molar equivalents) were weighed and placed in a reaction vessel, and then the atmosphere in the reaction vessel was replaced by a nitrogen gas. In the reaction vessel, THF (2.0 ml, anhydrous, free from stabilizer) was added, followed by stirring at room temperature overnight. Under a nitrogen atmosphere, NiCl₂/2,9-dimethyl-1,10-phenanthroline complex (4.3 mg, 0.0126 mmol, 0.10 molar equivalents) was added, followed by stirring at room temperature for 30 minutes. To the resultant reaction solution, a THF solution (5 ml) of ER-804030 (200 mg) and chlorotrimethylsilane (15.0 mg, 0.139 mmol, 1.1 molar equivalents) were added in turn, followed by stirring at room temperature for 3 hours. After confirming the disappearance of ER-804030 by HPLC, the reaction solution was cooled in ice bath, and then hydrochloric acid aqueous solution (0.5 N, 6.0 ml) was added. After stirring for 50 minutes, hexane (7.0 ml) was added to the reaction solution, followed by stirring for 5 minutes, and then the aqueous layer was isolated under a nitrogen atmosphere. Under a nitrogen atmosphere, the aqueous layer was extracted with heptane (2.0 ml), followed by mixing with the organic layer, and washing with potassium carbonate aqueous solution (20% by weight, 2.0 ml). The organic layer was concentrated and subjected to azeotropic drying with ethyl acetate. HPLC analysis was conducted on the resultant product using MTBE solution. As a result, the yield was 94.0% (HPLC quantitative yield).

### Example 5

### Production Example 1 of ER-118047/048

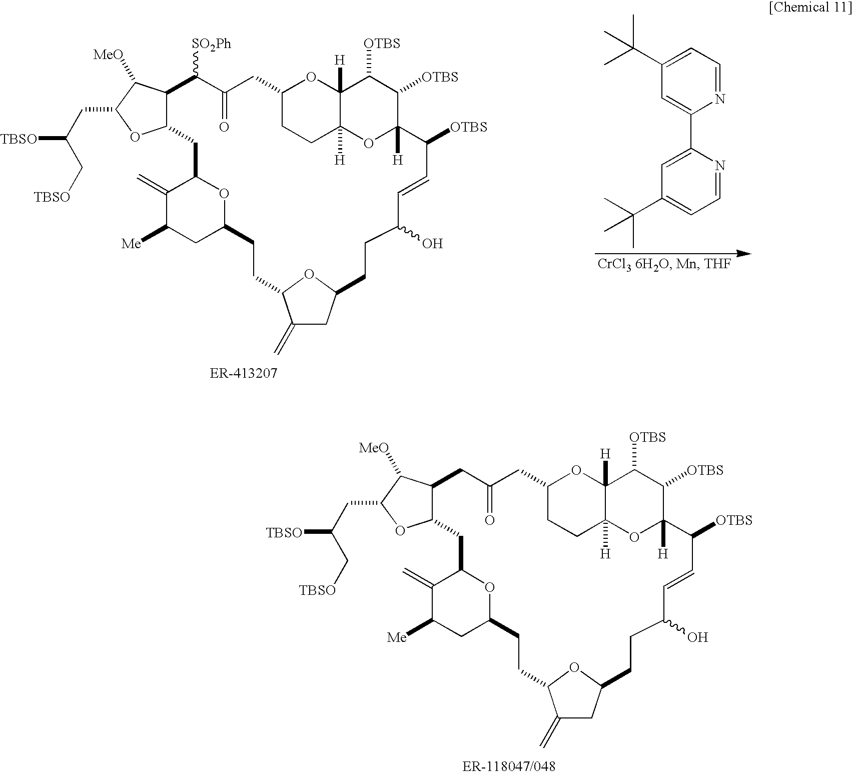

In a reaction vessel, under an argon atmosphere, THF (1 mL) was added to a solid mixture of ER-413207 (50.4 mg, purity: 93.7% by weight, 0.0323 mmol), 4,4′-di-t-butyl-2,2′-bipyridyl (10.2 mg, 0.0382 mmol), CrCl₃.6H₂O (11.0 mg, 0.0413 mmol) and powdered manganese (10.1 mg, 0.184 mmol) at room temperature (21.2° C.), followed by stirring for one hour. After terminating the reaction by adding heptane (about 1 mL) to the reaction mixture, methanol (about 1 mL) was added and the reaction mixture was further stirred for 20 minutes. The reaction mixture was concentrated and methanol was added again, followed by stirring and further concentration to obtain the objective compound ER-118047/048 as a diastereomer mixture. The resultant crude product was quantitatively determined by a HPLC external standard method to determine the yield. As a result, the yield was 93.6%. The crude product was purified by silica gel column chromatography (eluate: heptane/ethyl acetate) to obtain a purified product as a colorless solid.

¹H NMR (400 MHz, CDCl₃) 6.06 (dd, 1H, J=16.4, 7.2 Hz), 5.75 (dd, 1H, J=15.6, 4.4 Hz), 4.95 (s, 2H), 4.89 (s, 1H), 4.78 (s, 2H), 4.24 (brs, 2H), 4.06 (s, 1H), 4.04-3.98 (m, 1H), 3.94-3.68 (m, 7H), 3.63-3.52 (m, 3H), 3.47 (dd, 1H, J=10.4 Hz, J=5.2 Hz), 3.41 (d, 1H, J=3.6 Hz), 3.26 (s, 3H), 2.90 (dd, 1H, J=9.6 Hz, 2.4 Hz), 2.80 (dd, 1H, J=15.6 Hz, 6.4 Hz), 2.68-2.44 (m, 4H), 2.40-2.18 (m, 3H), 2.00 (t, 2H, J=6.0 Hz), 1.98-1.20 (m, 17H), 1.07 (d, 3H, J=6.4 Hz), 0.95 (s, 9H), 0.92 (s, 9H), 0.87 (s, 9H), 0.87 (s, 9H), 0.83 (s, 9H), 0.12 (s, 6H), 0.11 (s, 3H), 0.09 (s, 3H), 0.06 (s, 3H), 0.05 (s, 3H), 0.03 (s, 3H), 0.02 (s, 3H), 0.01 (s, 3H), −0.01 (s, 3H) MS m/z 1344 (M+23)

### Example 6

### Production Example 2 of ER-118047/048

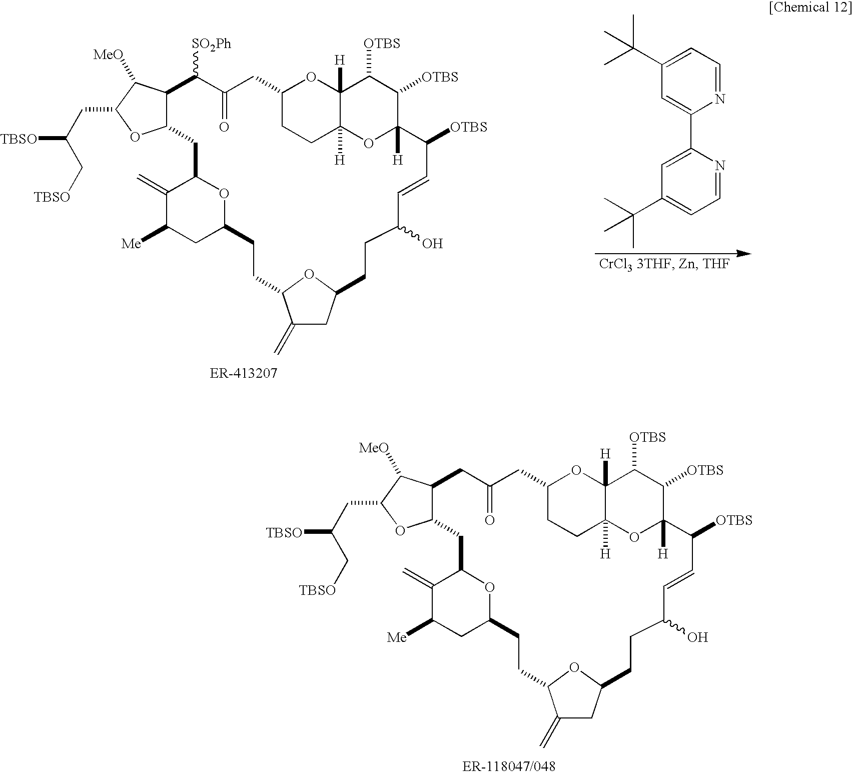

In a reaction vessel, under an argon atmosphere, THF (0.3 mL) was added to a solid mixture of ER-413207 (10.1 mg, purity: 85.0% by weight, 0.00587 mmol), 4,4′-di-t-butyl-2,2′-bipyridyl (11.0 mg, 0.0410 mmol), CrCl₃.3THF (15.4 mg, 0.0411 mmol) and powdered zinc (8.95 mg, 0.137 mmol) at room temperature (around 23° C.) and then the reaction mixture was stirred for about 19 hours. After terminating the reaction by adding heptane (about 0.5 ml) to the mixture, the reaction mixture was analyzed by a HPLC external standard method and the objective product was quantitatively determined thereby determining the yield of the objective product. As a result, the yield was 88.7% (diastereomer mixture).

### Example 7

### Production Example 3 of ER-118047/048

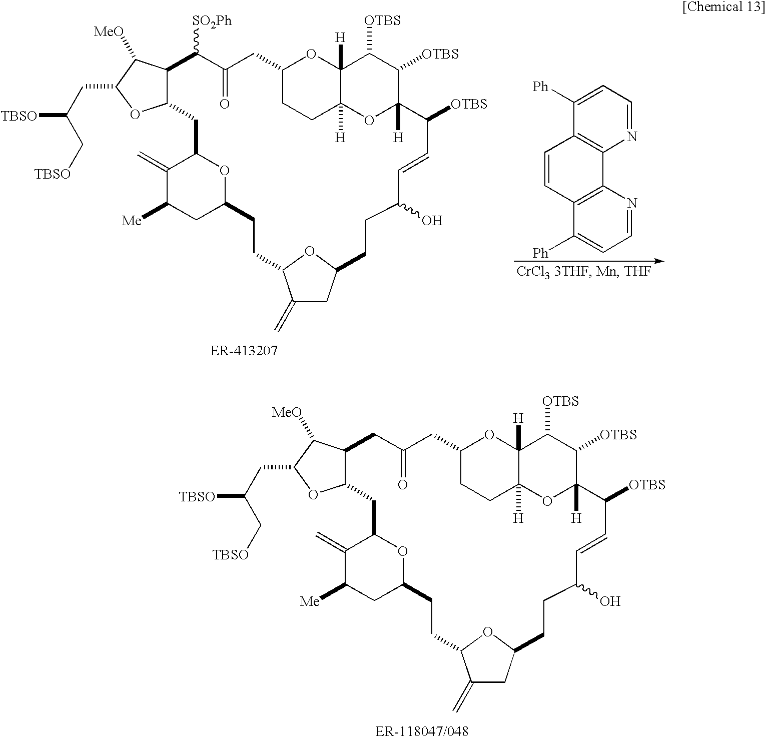

In a flask, under an argon atmosphere, THF (0.3 mL) was added to a solid mixture of ER-413207 (10.4 mg, 87.5% by weight, 0.00622 mmol), 4,7-diphenyl-1,10-phenanthroline (Bathophenanthroline) (15.1 mg, 0.0454 mmol), CrCl₃.3THF (17.0 mg, 0.0454 mmol) and powdered manganese (8.31 mg, 0.1513 mmol) at room temperature (around 23° C.) and the resultant reaction mixture was stirred for about 14 hours. After terminating the reaction by adding heptane (about 0.5 ml) to the reaction mixture, the reaction mixture was analyzed by a HPLC external standard method and the objective product was quantitatively determined thereby determining the yield of the objective product. As a result, the yield was more than 99% (diastereomer mixture).

### Example 8

### Production Example 4 of ER-118047/048

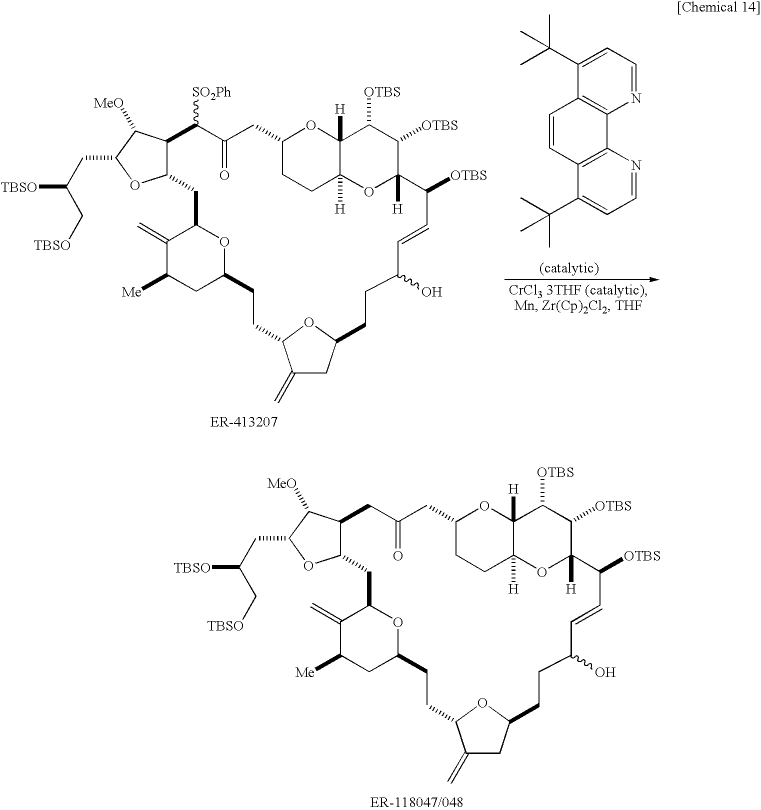

In a reaction vessel, under an argon atmosphere, THF (1 mL) was added to a solid mixture of ER-413207 (49.9 mg, 85.0% by weight, 0.0290 mmol), 4,4′-di-tert-butyl-2,2′-bipyridyl (1.84 mg, 0.0068 mmol), CrCl₃.3THF (2.56 mg, 0.0068 mmol), dicyclopentadienylzirconium dichloride (Cp₂ZrCl₂) (12.0 mg, 0.0410 mmol) and powdered manganese (9.39 mg, 0.171 mmol) at room temperature (around 23° C.) and the resulting reaction mixture was stirred for about 14 hours. After terminating the reaction by adding heptane (about 1 ml) to the reaction mixture, the reaction mixture was analyzed by a HPLC external standard method and the objective product was quantitatively determined thereby determining a yield of the objective product. As a result, a yield was more than 90.8% (diastereomer mixture).

### Example 9

### Production Example 5 of ER-18047/048

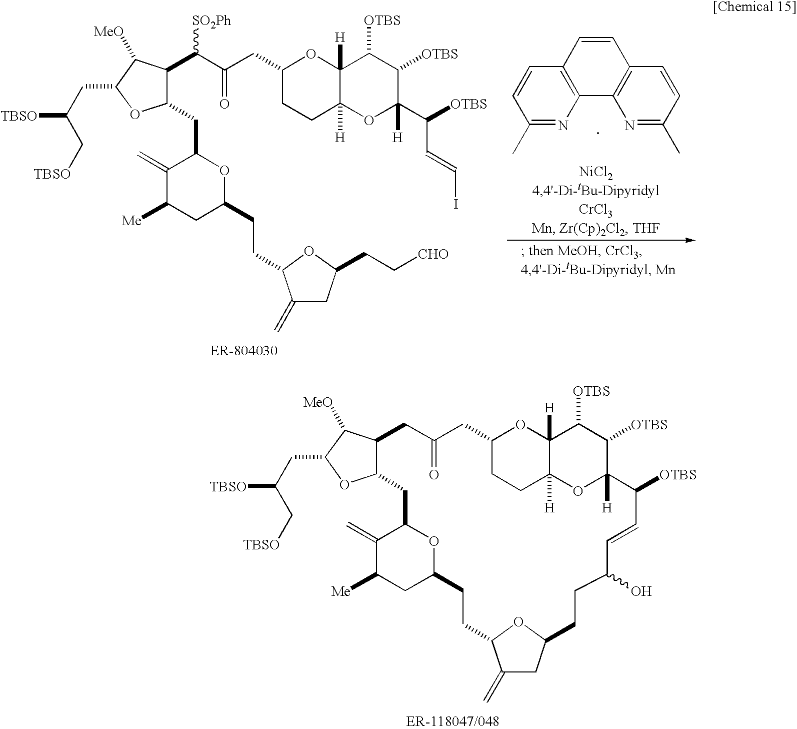

4,4′-di-t-butyl-bipyridyl (10.1 mg, 0.0378 mmol, 0.10 molar equivalents), CrCl₃ (6.0 mg, 0.0378 mmol, 0.10 molar equivalents), a manganese powder (83.0 mg, 1.51 mmol, 4.0 molar equivalents) and bis(cyclopentadienyl)zirconium dichloride (122 mg, 0.416 mmol, 1.1 molar equivalents) were weighed and placed in a reaction vessel, and then the atmosphere in the reaction vessel was replaced by a nitrogen gas. In the reaction vessel, THF (6.0 ml, anhydrous, free from stabilizer) was added, followed by stirring at room temperature for 3 hours. Under a nitrogen atmosphere, NiCl₂/2,9-dimethyl-1,10-phenanthroline complex (12.8 mg, 0.0378 mmol, 0.10 molar equivalents) was added to this reaction solution, followed by stirring at room temperature for 30 minutes. To the resultant reaction solution, a THF solution (15 ml) of ER-804030 (600 mg) was added through 15 minutes, followed by stirring at room temperature for 2 hours. After confirming the disappearance of ER-804030 by HPLC, methanol (76.4 μL, 1.89 mmol, 5.0 molar equivalents), manganese powder (125 mg, 2.27 mmol, 6.0 molar equivalents), 4,4′-di-t-butyl-bipyridyl (203 mg, 0.756 mmol, 2.0 molar equivalents) and CrCl₃ (120 mg, 0.756 mmol, 2.0 molar equivalents) were added in turn to the reaction solution. After stirring the reaction solution at room temperature overnight, the disappearance of ER-413207 was confirmed by HPLC, and heptane (21.0 ml) and methanol (9.0 ml) were added and then stirred for 15 minutes. Under a nitrogen atmosphere, the reaction solution was washed twice with hydrochloric acid aqueous solution (0.5 N, 18.0 ml, 6.0 ml) in a separate solution. Under a nitrogen atmosphere, the mixed aqueous layer was reextracted with heptane (6.0 ml). The reextracted heptane layer was mixed with the organic layer, followed by adding potassium carbonate aqueous solution (5% by weight, 9.0 ml), washing with the potassium carbonate aqueous solution, and then separating the solution. The organic layer was concentrated and subjected to azeotropic drying with ethyl acetate. HPLC analysis was conducted on the resultant product using MTBE solution. After HPLC analysis, the MTBE solution was concentrated to obtain ER-1118047/048 crude product 513.9 mg. As a result, the yield was 85.1% (HPLC quantitative yield; diastereomer mixture).

### Example 10

### Production Example of ER-118046

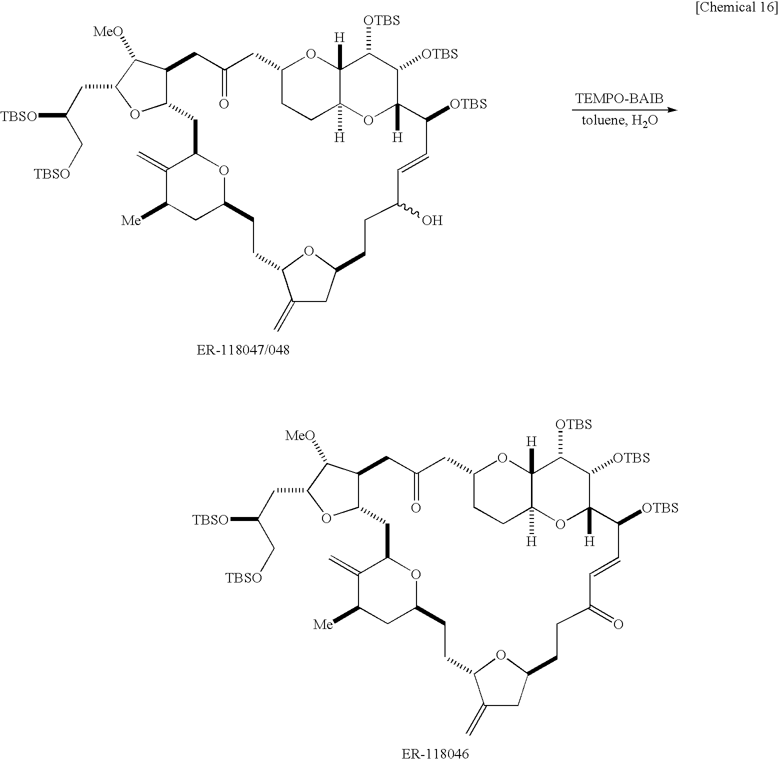

In a reaction vessel, to a solid mixture of ER-118047/048 (50.3 mg, 97.2% by weight, 0.0377 mmol) and (diacetoxyiodo)benzene (30.5 mg, 0.0945 mmol), a preliminarily prepared toluene solution (0.0378 M, 0.5 mL) of TEMPO (2,2,6,6-tetramethyl-1-piperidinyloxyl, free radical) was added at room temperature (25° C.) and H₂O (17 μL, 0.945 mmol) was further added, and then the resultant reaction solution was stirred for about 20 hours. The yield of the objective product in the reaction solution was determined by quantitative determination using a HPLC external standard method. As a result, the yield was 92.6%. The crude product was purified by silica gel column chromatography (eluate: heptane/MTBE) to obtain a purified product as a colorless solid.

¹H NMR (400 MHz, CDCl₃) 6.33 (d, 1H, J=16.4 Hz), 5.03-4.93 (m, 2H), 4.87 (s, 1H), 4.82 (s, 1H), 4.77 (s, 1H), 4.22 (brs, 1H), 4.10-3.98 (m, 3H), 3.91-3.74 (m, 5H), 3.68 (m, 1H), 3.55 (dd, 2H, J=10.4, 5.2 Hz), 3.47 (dd, 1H, J=10.4, 5.2 Hz), 3.43-3.36 (m, 2H), 3.29 (s, 3H), 2.93 (dd, 1H, J=9.6, 2.4 Hz), 2.84 (dd, 1H, J=15.6, 7.2 Hz), 2.77-2.58 (m, 4H), 2.55-2.40 (m, 3H), 2.32-2.19 (m, 2H), 2.03 (dd, 1H, J=12.8, 7.6 Hz), 1.98-1.18 (m, 16H), 1.06 (d, 3H, J=6.4 Hz), 0.96 (s, 9H), 0.93 (s, 9H), 0.87 (s, 9H), 0.86 (s, 9H), 0.86 (s, 9H), 0.18 (s, 3H), 0.13 (s, 3H), 0.11 (s, 6H), 0.06 (s, 3H), 0.04 (s, 3H), 0.03 (s, 3H), 0.02 (s, 6H), −0.06 (s, 3H) MS m/z 1342 (M+23)

1. A compound represented by formula (I) shown below: 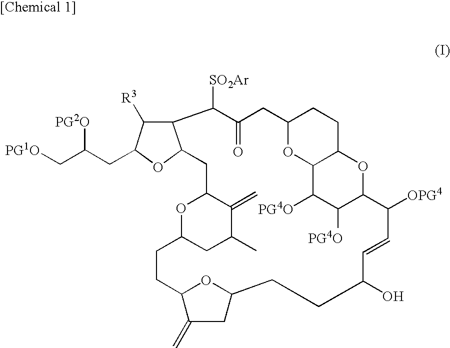
wherein R³ represents R or OR, and R represents a hydrogen atom, a halogen atom, a C₁₋₄ halogenated aliphatic group, benzyl, or a C₁₋₄ aliphatic group; Ar represents a substituted or unsubstituted aryl group, or a substituted or unsubstituted heteroaryl group; and PG¹, PG² and PG⁴ each independently represents a protective group of a hydroxyl group.

2. A method for producing a compound represented by formula (III) shown below: 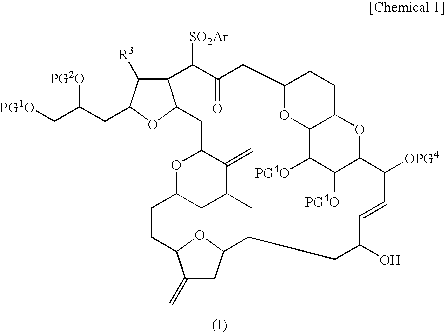
wherein R³, PG¹, PG² and PG⁴ are as defined in formula (I) shown below,
which comprises treating a compound represented by formula (I) shown below: 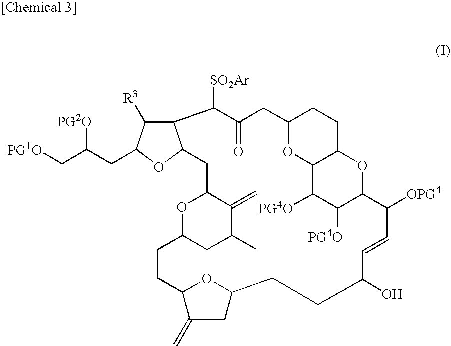
wherein R³ represents R or OR, and R represents a hydrogen atom, a halogen atom, a C₁₋₄ halogenated aliphatic group, benzyl, or a C₁₋₄ aliphatic group; Ar represents a substituted or unsubstituted aryl group, or a substituted or unsubstituted heteroaryl group; and PG¹, PG² and PG⁴ each independently represents a protective group of a hydroxyl group,
with a trivalent chromium compound and at least one kind of metal selected from the group consisting of manganese and zinc in a solvent in the presence of a ligand represented by formula (II) shown below: 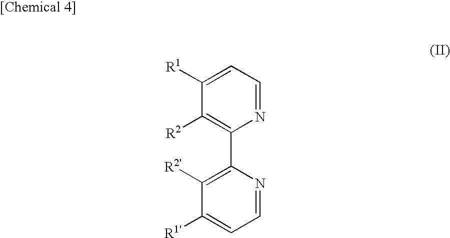
wherein R¹ and R^{1′} each independently represents a C₃₋₁₂ alkyl group, or an unsubstituted or substituted phenyl group; and R² and R^{2′} each independently represents a hydrogen atom or a C₁₋₆ alkyl group, or R² and R^{2′} may be combined to form a fused ring together with a pyridine ring to which they are attached.

3. The method according to claim 2, wherein the trivalent chromium compound is Cr(III) X₃, in which X represents a halogen atom.

4. The method according to claim 3, wherein X is Cl or Br.

5. The method according to claim 3, wherein the trivalent chromium compound is at least one kind selected from the group consisting of CrCl₃ anhydride, CrCl₃.6H₂O and CrCl₃.3THF.

6. The method according to claim 2, wherein R¹ and R^{1′} in the formula (II) are t-butyl, phenyl or nonyl, and R² and R^{2′} are hydrogen atoms, or R² and R^{2′} are combined to form a fused ring together with a pyridine ring to which they are attached.

7. The method according to claim 2, wherein a metallocene compound selected from the group consisting of Ti, Zr and Hf compounds, containing a cyclopentadienyl ring, is further added.

8. The method according to claim 2, wherein said treatment is carried out at 20 to 30° C.

9. The method according to claim 2, wherein the solvent is a mixture of one or more kinds selected from the group consisting of tetrahydrofuran, dimethoxyethane, methyl t-butylether, dimethylformamide, methanol and acetonitrile.
